(19) United States
(12) Patent Application Publication
(10) Pub. No.: US 2026/0194534 A1
(43) Pub. Date: Jul. 9, 2026

(54) METHODS, SYSTEMS, AND COMPUTER READABLE MEDIA FOR PREDICTING CROSS-MATCH COMPATIBILITY USING MACHINE LEARNING HUMAN LEUKOCYTE ANTIGEN (HLA) ANTIBODY MODEL

(71) Applicant: The University of North Carolina at Chapel Hill, Chapel Hill, NC (US)

(72) Inventors: Eric Thomas Weimer, Durham, NC (US); Katherine Alta Newhall, Carrboro, NC (US)

(21) Appl. No.: 19/554,354

(22) Filed: Mar. 2, 2026

Related U.S. Application Data
(60) Provisional application No. 63/766,107, filed on Mar. 3, 2025.

(51) Int. Cl. G01N 33/68; G16B 30/00

(52) U.S. Cl. CPC G01N 33/6854; G16B 30/00; G01N 2800/245

(57) ABSTRACT

A method for predicting a virtual crossmatch outcome using a machine learning human leukocyte antigen (HLA) antibody model includes receiving, as input, donor specific antibody (DSA) mean fluorescence intensity (MFI) values obtained from mixing donor-specific single antigen beads with serum samples of a prospective transplant recipient. The method further includes normalizing the DSA MFI values. The method further includes inputting the normalized DSA MFI values into the machine learning HLA antibody model. The method further includes receiving, as output from the HLA antibody model, a predicted crossmatch probability. The method further includes using the predicted crossmatch probability to inform a transplant decision.

### PRIORITY CLAIM

This application claims the priority benefit of U.S. Provisional Patent Application Ser. No. 63/766,107 filed Mar. 3, 2025, the disclosure of which is incorporated herein by reference in its entirety.

### TECHNICAL FIELD

The subject matter described herein relates to virtual crossmatch testing. More particularly, the subject matter described herein relates to methods, systems, and computer readable media for predicting cross-match compatibility using a machine learning HLA antibody model.

### BACKGROUND

In the field of human immunology, crossmatch testing is used to determine a likelihood that a prospective tissue recipient will reject tissue from a donor. A physical crossmatch test involves incubating lymphocytes of a tissue donor in serum obtained from a prospective tissue recipient to determine whether the recipient has antibodies to human leukocyte antigens (HLAs) of the donor. A physical crossmatch test is accurate but requires incubation of the lymphocytes of the donor in serum of each prospective recipient, making the test non-scalable to screen large numbers of prospective tissue recipients against a donor. In addition, for some organ transplants, such as heart and lung transplants, physical crossmatching is not available to determine donor-recipient compatibility, because the organs only remain viable for a transplant for a short time period after being removed from the donor, and that time period is insufficient for physical crossmatching.

Due to the time required for physical crossmatching and instances in which physical crossmatching is not available, virtual crossmatching is used to inform transplant decisions. Virtual crossmatching is performed by mixing synthetic beads coated with individual HLA antigens with prospective recipient serum and using flow cytometry to detect the HLA antibodies present in the recipient serum. The HLA antibodies present in the sera of different recipients are stored in a database and subsequently compared to HLA typing data of tissue donors to determine compatibility.

Virtual crossmatching is more scalable than physical crossmatching because serum from prospective tissue recipients can be tested once to determine the HLA antibodies present, and that data can be stored in a database and “virtually” compared against HLA typing data of different donors. As a result, virtual crossmatch testing can be used to screen an entire database of prospective tissue recipients against a given donor's HLA typing data.

One problem with conventional virtual crossmatching is that the interpretation of crossmatching results is subject to human cognitive bias, such as recency bias, which may affect the transplant decision. For example, one technique for interpreting virtual crossmatching results is to sum HLA donor specific antibody (DSA) mean fluorescence intensity (MFI) values obtained from flow cytometric testing. The sum of the HLA DSA MFI values is compared to a threshold. If the sum of the HLA DSA MFI values is above the threshold, the clinician may determine that a transplant should not occur. If the sum of the MFI values is below the threshold, the clinician may determine that the transplant should occur. The setting of the DSA MFI threshold is subjective and may be influenced by cognitive bias. In addition, some DSA MFI values may be more important than others in predicting the immune system's reaction to a particular transplant, and simply summing the DSA MFI values does not reflect the relative importance of the different DSA MFI values.

Another problem associated with virtual crossmatching is that HLA alleles of the donor and the recipient may not be known, for example, due to the limited time available to make an organ transplant decision. Determining HLA alleles of the donor and the recipient requires DNA sequencing, which can be more time consuming than serologic typing. Another problem with virtual crossmatching is that there are many complex biological interactions between a recipient's immune system and a donor organ that have been studied individually, but that have not been considered together as part of a unified crossmatching evaluation model. Such interactions include HLA antibodies, HLA genotype, donor HLA expression, HLA antibody avidity, and HLA eplets.

In light of these and other difficulties, there exists a need for methods, systems, and computer readable media for predicting cross-match compatibility using know and imputed alleles and a machine learning HLA antibody model.

### SUMMARY

A method for predicting a virtual crossmatch outcome using a machine learning human leukocyte antigen (HLA) antibody model includes receiving, as input, DSA MFI values for donor-specific HLA gene pairs obtained from mixing donor-specific single antigen beads with serum samples of a prospective transplant recipient. The method further includes normalizing the DSA MFI values. The method further includes inputting the normalized DSA MFI values into the machine learning HLA antibody model. The method further includes receiving, as output from the HLA antibody model, a predicted crossmatch probability. The method further includes using the predicted crossmatch probability to inform a transplant decision.

According to another aspect of the subject matter described herein, the method for predicting a virtual crossmatch outcome includes identifying donor-specific HLA gene pairs, where the DSA MFI values are DSA MFI values for the donor-specific HLA gene pairs and further comprising, for each gene pair, selecting a higher of two DSA MFI values.

According to another aspect of the subject matter described herein, identifying the donor-specific HLA gene pairs comprises identifying the donor-specific HLA gene pairs from DNA sequencing of an HLA system of a donor. The method of claim 2 wherein identifying the donor-specific HLA gene pairs comprises imputing the donor-specific HLA gene pairs from HLA serology data of the donor.

According to another aspect of the subject matter described herein, the method for predicting a virtual crossmatch outcome includes identifying recipient HLA gene pairs.

According to another aspect of the subject matter described herein, the method for predicting a virtual crossmatch outcome includes reducing DSA MFI values for donor HLA gene pairs that match recipient HLA gene pairs. The method of claim 1 comprising identifying donor-specific HLA alleles and wherein the DSA MFI values are DSA MFI values for the donor-specific HLA alleles.

According to another aspect of the subject matter described herein, the method for predicting a virtual crossmatch outcome includes identifying donor-specific antigens encoded by donor-specific HLA gene pairs, wherein the DSA MFI values are DSA MFI values for the donor-specific antigens.

According to another aspect of the subject matter described herein, inputting the normalized DSA MFI values into the machine learning HLA antibody model includes inputting the normalized DSA MFI values into a HLA antibody model trained to predict the crossmatch probability from known transplant outcome data.

According to another aspect of the subject matter described herein, receiving the predicted crossmatch probability includes receiving a predicted positive crossmatch probability indicating a likelihood that a transplant will not be successful.

According to another aspect of the subject matter described herein, using the predicted crossmatch probability to inform the transplant decision includes determining whether or not to perform a transplant for a donor-recipient pair based on the predicted crossmatch probability.

According to another aspect of the subject matter described herein, a a system for predicting a virtual crossmatch outcome using a machine learning human leukocyte antigen (HLA) antibody model is provided. The system includes a computing platform including at least one processor and a memory. The system further includes a data preparation module implemented by the at least one processor for receiving, as input, donor specific antibody (DSA) mean fluorescence intensity (MFI) values obtained from mixing donor-specific single antigen beads with serum samples of a prospective transplant recipient and normalizing the DSA MFI values. The system further includes an HLA antibody model implemented by the at least one processor for receiving, as input, the normalized DSA MFI values and generating, as output, a predicted crossmatch probability.

According to another aspect of the subject matter described herein, the data preparation module is configured to identify donor-specific HLA gene pairs, where the DSA MFI values are DSA MFI values for the donor-specific HLA gene pairs and the data preparation module is configured to, for each gene pair, selecting a higher of two DSA MFI values.

According to another aspect of the subject matter described herein, the data preparation module is configured to identify the donor-specific HLA gene pairs from DNA sequencing of an HLA system of a donor

According to another aspect of the subject matter described herein, the data preparation module is configured to identify the donor-specific HLA gene pairs by imputing the donor-specific HLA gene pairs from HLA serology data of the donor.

According to another aspect of the subject matter described herein, the data preparation module is configured to identify recipient HLA gene pairs.

According to another aspect of the subject matter described herein, the data preparation module is configured to reduce DSA MFI values for donor HLA gene pairs that match recipient HLA genes pairs.

According to another aspect of the subject matter described herein, the data preparation module is configured to identify donor-specific HLA alleles and the DSA MFI values are DSA MFI values for the donor-specific HLA alleles.

According to another aspect of the subject matter described herein, the data preparation module is configured to identify donor-specific antigens encoded by donor-specific HLA gene pairs, wherein the DSA MFI values are DSA MFI values for the donor-specific antigens.

According to another aspect of the subject matter described herein, the HLA antibody model is trained to predict the crossmatch probability from known transplant outcome data.

According to another aspect of the subject matter described herein, the predicted crossmatch probability comprises a predicted positive crossmatch probability indicating a likelihood that a transplant will not be successful.

According to another aspect of the subject matter described herein, a non-transitory computer readable medium having stored thereon executable instructions that when executed by a processor of a computer control the computer to perform steps is provided. The steps include receiving, as input, donor specific antibody (DSA) mean fluorescence intensity (MFI) values obtained from mixing donor-specific single antigen beads with serum samples of a prospective transplant recipient. The steps further include normalizing the DSA MFI values. The steps further include inputting the normalized DSA MFI values into a machine learning donor-specific human leukocyte antigen (HLA) antibody model. The steps further include receiving, as output from the HLA antibody model, a predicted crossmatch probability. The steps further include using the predicted crossmatch probability to inform a transplant decision.

The subject matter described herein may be implemented in hardware, software, firmware, or any combination thereof. As such, the terms “model”, “function”, “node”, or “module”, as used herein, refer to hardware, which may also include software and/or firmware components, for implementing the feature being described. In one exemplary implementation, the subject matter described herein may be implemented using a computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

The subject matter described herein will now be explained with reference to the accompanying drawings of which:

FIG. 1 illustrates an overview of machine learning models used to generate the virtual crossmatch risk probability;

FIGS. 2A-2C illustrate the performance of the machine learning models under different sampling conditions;

FIGS. 3A and 3B illustrate the effect of HLA antibodies in ML model prediction;

FIG. 4 illustrates ML model performance;

FIG. 5 illustrates an example of HLA antibody model inputs and outputs for a potential transplant recipient X and a donor Y;

FIG. 6 is a block diagram illustrating an example of a trained machine learning model and its use to generate crossmatch probability values; and

FIG. 7 is a flow chart illustrating an exemplary process for predicting a virtual crossmatch outcome using a machine learning HLA antibody model.

### DETAILED DESCRIPTION

The subject matter described herein includes a study whose purpose was to enhance the prediction of solid-organ recipient and donor crossmatch compatibility by applying machine learning (ML). Prediction of crossmatch compatibility is complex and requires an understanding of the recipient and donor human leukocyte antigen (HLA) alleles and recipient HLA antibodies. An HLA allele imputation system that converts HLA antigens to alleles was developed to enhance the prediction's performance. The imputed and known HLA alleles were combined for recipient and donor with a recipient's HLA antibody profile. After processing, donor-specific antibodies were input into various ML models. Next, an ML model was developed and characterized based on determining donor-specific antibodies using the full HLA antibody profile of the recipient without laboratory interpretation. The models achieved a receiver operating characteristic area under the curve (ROC-AUC) of 0.975. These results demonstrate that the models can predict crossmatch reactivity and yield insight into the importance of specific HLA antibodies in the transplant-matching process. These data represent our understanding of personalized histocompatibility risk assessments.

### INTRODUCTION

Solid-organ transplantation is a life-saving treatment for many patients with chronic diseases. Currently, more than 114,000 patients are waiting to receive an organ. Kidney transplants are the most common type of transplant worldwide, with over 27,332 performed in the United States in 2023, representing a 7.18% increase from 2022. Advances in transplantation have reduced the risk of hyperacute allograft rejection by assessing a recipient's HLA antibody status and using physical crossmatching. Physical crossmatching is a test that examines the interaction between a recipient's serum and the donor's lymphocytes. A positive reaction indicates pre-existing immunity to the donor, usually due to pre-formed anti-HLA antibodies, and is generally considered a contraindication for kidney transplant (Tambur et al. 2018).

A virtual crossmatch (VXM) requires, at a minimum, knowledge of a recipient's HLA antibody status and HLA genotyping for both the recipient and donor (Roll et al. 2020; Eby et al. 2016; Amico et al. 2009). These factors and others are used to determine the relative immunologic risk of a potential transplant pair. Current practice is to further evaluate pair compatibility by a physical flow cytometric crossmatch (XM) involving the recipient's serum and the donor's lymphocytes (T and B cells) to identify potential hyperacute allograft rejection (Takeda et al. 2000; Downing 2012; Sullivan et al. 2018; Liwski et al. 2018). Both the virtual and physical crossmatches are time-consuming, as they are largely done by hand, leading to increased ischemic organ time. Additionally, the demand for VXM has dramatically increased recently due to changes in the deceased donor service area, as a potential deceased donor is available for a larger pool of recipients (Morris et al. 2010; Morris et al. 2019). Within the transplant community, there is active debate about the use of VXM, the need for physical crossmatching, and the use of HLA eplets in matching or organ allocation. There have been attempts to devise various models to understand the complex relationship between HLA antibodies and their respective targets on donor cells (Weimer and Newhall 2019; Norin et al. 2022; Valentin et al. 2023; Wrenn et al. 2018; Johnson et al. 2016). Previous work has shown that manual VXM correctly predicts 89-97% of flow cytometric crossmatches (Perssaari et al. 2018; Sullivan et al. 2018; Piazza et al. 2014; Chowdhry et al. 2020; Weimer and Newhall 2019; Olszowska-Zaremba et al. 2022). However, that number is drastically lower for highly sensitized patients (Olszowska-Zaremba et al. 2022; Morris et al. 2010). Our previous work established that mathematical modeling could yield powerful insights into crossmatch prediction and transplant biology (Weimer and Newhall 2019). However, to enable a deeper understanding of transplant immunologic compatibility more advanced methods are required. For example, several studies have shown that donor HLA expression impacts physical crossmatch outcomes (Badders et al. 2015; Montgomery et al. 2020).

Machine learning (ML) is well suited for complex biological interactions in which patterns are not readily observable, and mechanisms are not fully characterized. Currently, most studies have applied ML to six areas of transplantation: radiological or pathological evaluation, prediction of allograft survival, immunosuppression optimization, diagnosis of allograft rejection, and prediction of early allograft function (Miller et al. 2022; Tian et al. 2023; Mark et al. 2019; Becker et al. 2020). The immunologic complexity of transplant pairs, therefore, lends itself to ML. There are numerous interactions between a recipient's immune system and the donor organ. Some of the known interactions include HLA antibodies, HLA genotype, donor HLA expression, HLA antibody avidity, and HLA eplets. By combining ML with these known biological connections, observations previously poorly understood can be identified. This is particularly important for the application of HLA antibodies in the context of pre-transplant risk assessments.

To yield deeper insights into immunologic compatibility, we have developed and evaluated a digital alloimmune risk assessment (DARA) tool that utilizes machine learning with HLA antibodies to predict physical crossmatch compatibility. As the optimal determination of donor-specific antibodies (DSA) requires high-resolution HLA typing for both the recipient and donor (Senev et al. 2021), our DARA tool includes an HLA allele imputation system to determine HLA alleles (if unknown) from HLA serologic-level typing. It also consists of the estimation of physical crossmatch outcomes using machine learning models. This approach modernizes the current standard of care approach of using curated recipient HLA antibody profiles and the HLA genotype of the potential donor.

### Results

### Overview of Machine Learning Models

To develop an unbiased, independent algorithm for crossmatch prediction, we analyzed flow cytometric crossmatches (XM) performed at UNC Hospitals from January 2015 to June 2024. XM cases with unexplained results or known false reactivity were excluded from the study (see Materials and Methods for detailed exclusion criteria).

Our HLA antibody model automatically detected donor-specific HLA antibodies (DSAs) from the complete HLA antibody profile of each recipient. The mean fluorescence intensities (MFIs) of these allele-specific DSAs were used as features for machine learning (ML) training. Multiple ML models were evaluated, with the model yielding the highest custom metric score selected for subsequent comparative analyses.

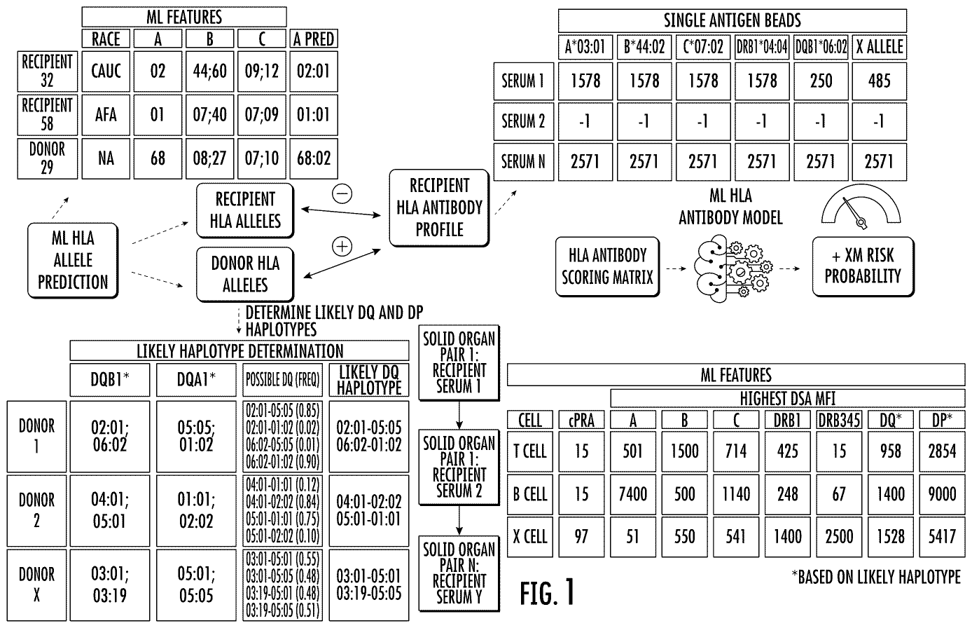

For model training, we implemented the following:
1. Data Split: 75% of the XM cases were used for training, stratified based on recipient and XM result. This approach maintained a consistent ratio of positive XM between the training and test sets (FIG. 1). This approach also prevented any data leakage based on the recipient HLA antibodies and maximized data usage.
2. Training Dataset: The HLA antibody model was trained on 9,892 XM cases, comprising of:
   9,292 negative cases (93.93%)
   600 positive cases (6.07%)
3. Cross-Validation: To prevent overfitting, five-fold cross-validation was used during model fitting and while testing various hyperparameter sets. Early stopping was used where appropriate.
4. Hyperparameter Selection: The hyperparameter set yielding the highest custom metric score was selected for the final model.
5. Threshold Optimization: Using the established hyperparameters, the optimal threshold for the f1-weighted score was determined. This optimized threshold was then applied in subsequent analyses.

FIG. 1 illustrates an overview of machine learning models used to generate the virtual crossmatch risk probability. The ML models begin with HLA allele imputation for either recipients or donors. The imputed HLA alleles are then used to determine donor-specific antibodies (DSAs) in the HLA antibody model. The HLA antibody mean fluorescent intensity (MFI) is identified using the donor-specific HLA alleles from single-antigen bead testing and used to generate an HLA antibody scoring matrix. The scoring matrix used for ML training (75% of total data) using the optimal hyperparameters that generated the highest custom scorer using five-fold cross-validation. Each trained ML model was tested with an independent dataset (25% of data) using their optimal parameters to determine the positive crossmatch risk.

### Effect of HLA Allele Imputation on ML Model Performance

To enable use of historical data, HLA allele prediction ML models were developed. The models were specific to each HLA locus and yielded an internally validated average two-allele accuracy of 91.4%, greater than 99% with at least one allele accuracy. To assess the impact of HLA allele-level prediction on the performance of our HLA antibody ML model, we compared model performance with and without HLA imputation. The results of this comparison are summarized in Table 1. Imputation led to a decrease in the HLA antibody model's sensitivity by 6.3 percentage points (from 78.9% to 72.6%). There was no effect on the specificity. The Brier score loss decreased from 0.059 to 0.026 with imputation, indicating an increase in overall prediction performance. These observations are consistent with previous manual assessments of the impact of recipient and donor HLA alleles on donor-specific antibody evaluation and assignment. Together, these results support that the imputation accuracy does not detrimentally affect the accuracy of the downstream ML model.

TABLE 1
Effect of HLA allele imputation on ML model performance.

| | | | | | Brier | Total |
|---|---|---|---|---|---|---|
| | | | | Brier | Score | number of |
| Impu- | | | | Score | Loss per | Validation |
| tation | PPV | Sensitivity | Specificity | Loss | XM class | Pairs |
| No | 0.856 | 0.789 | 0.993 | 0.059 | Negative | 2604 |
| | | | | | XM: 0.001 | (5.6% + |
| | | | | | Positive | XM) |
| | | | | | XM: 0.073 | |
| Yes | 0.858 | 0.726 | 0.993 | 0.026 | Negative | 3285 |
| | | | | | XM: 0.023 | (6.8% + |
| | | | | | Positive | XM) |
| | | | | | XM: 0.027 | |

### HLA Antibody Machine Learning Model Characteristics

To address the imbalance in the dataset, where negative crossmatch (XM) results significantly outnumbered positive ones, we employed various sampling techniques. These techniques were applied only to the training data, while all models were evaluated using the same unmodified test data to ensure fair comparison. Synthetic Minority Oversampling Technique (SMOTE) was used to generate synthetic samples within the minority class (positive XM results). SMOTE creates new, synthetic examples in the feature space of the minority class, effectively increasing its representation in the dataset (Chawla et al. 2002). Random Undersampling randomly downsamples the majority class (negative XM cases). By reducing the number of negative XM cases, the technique aims to balance the dataset and potentially improve the model's ability to identify positive cases (Shao et al. 2024).

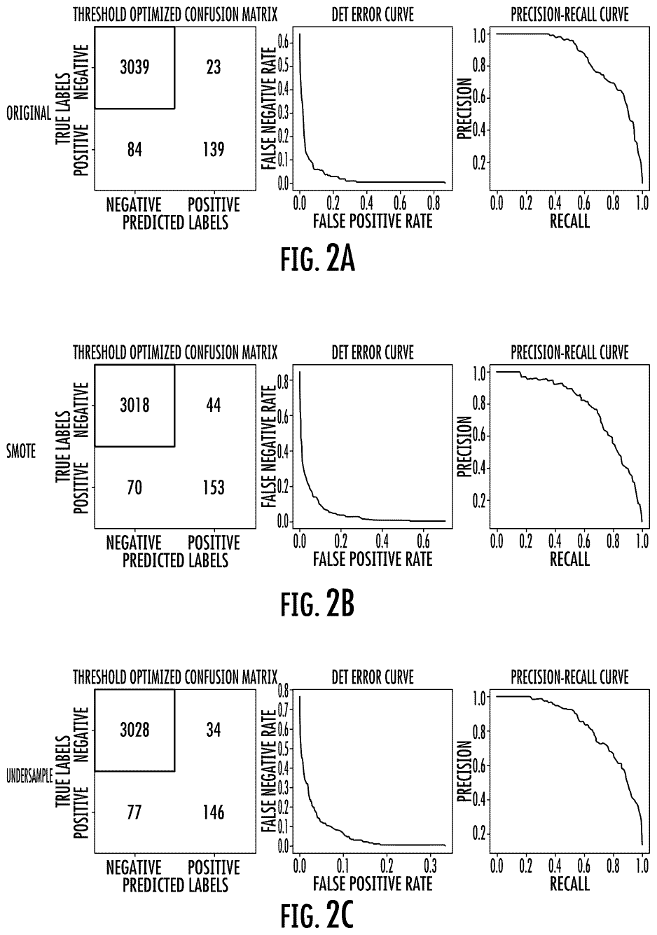

FIGS. 2A-2C illustrate the performance of the models under different sampling conditions. Overall, model performance was consistent across all the sampling techniques as evidenced by the similar shapes of the detection error tradeoff (DET) and precision-recall curves. This data indicates that the original model was relatively robust to class imbalance. The graphs in FIG. 2A illustrate results for the original unmanipulated dataset. FIG. 2B illustrates results obtained using Synthetic Majority Oversampling Techniques (SMOTE) to equalize the positive XM cases. FIG. 2C illustrates results for the random undersampling technique. The leftmost graphs in each row are confusion matrices. The center graphs in each row are detection error tradeoff (DET) graphs. The rightmost graphs in each row are precision-recall curves. All graphs were made using the optimal hyperparameter and optimal f1-weighted threshold ML model with the independent test dataset. All sampling techniques were only applied to training data.

### HLA Antibody-Based Machine Learning Model

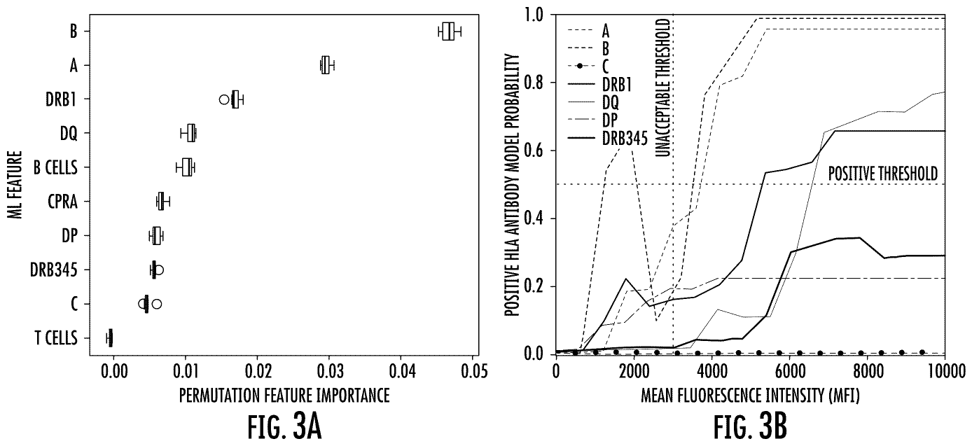

Having established the overall performance of our HLA antibody-based machine learning model, we next sought to understand how individual HLA antibodies contribute to the model's predictions. To achieve this, permutation feature importance, a model-agnostic technique that evaluates the importance of machine learning features (FIG. 3A) was used. This method assesses how the model's performance decreases when a single feature (DSA to a specific HLA loci) is randomly altered, providing insight into each antibody's relative impact on the model's predictions.

It is well established that the determination of HLA antibodies using solid-phase assays can lead to over-interpretation of a recipient's reactivity. There are several overreactive beads and HLA professionals often make manual interpretations of these complex reactivity patterns. To evaluate the impact of individual HLA antibodies on ML performance, a range of specific HLA antibody MFIs were analyzed. Specific HLA antibodies were incrementally increased from their minimum to maximum MFI value in the dataset with an absolute maximum of 10,000 MFI (FIG. 3B). These simulated the presence of a single donor-specific antibody. Each HLA locus-specific antibody's impact on ML prediction was assessed. Not surprisingly, increasing the antibody MFI value increased the likelihood of a positive crossmatch prediction for most of the loci.

The effect of HLA class I antibodies on ML prediction was assessed using only T cell responses, while HLA class II antibodies were assessed using B cell responses. (FIGS. 3A and 3B). Consistent with previous reports regarding HLA locus-specific expression, HLA-B, HLA-A, and HLA-DRB1 had the greatest effect on the model's prediction, further suggesting a connection between HLA protein expression and HLA antibody binding capacity (FIG. 3A) (Montgomery et al. 2020; Badders et al. 2015). Of note, HLA antibodies targeting HLA-C, HLA-DP, or HLA-DRB345 did not individually impact the prediction, suggesting a reliance on other HLA antibodies (FIGS. 3A and 3B). Consistent with the overreaction in the antibody assay, antibodies targeting HLA-DQ and -DRB1 required higher MFI values to affect the ML prediction (FIG. 3B). These observations are consistent with the over-reactivity of several HLA-DRB1 and HLA-DQ alleles in the HLA antibody assay and emphasize the need to list unacceptable HLA antibodies at the HLA antigen level. These data provide a data-driven approach to the individualized listing of unacceptable HLA antibodies. Additionally, the data demonstrate the feasibility of the models to learn representative transplant biology and aid in the understanding of global model predictions.

FIGS. 3A and 3B illustrate the effect of HLA antibodies in ML model prediction. FIG. 3A illustrates ML feature importance using the permutation technique. FIG. 3B illustrates stimulation of a single DSA and evaluation of the impact of various HLA antibodies on model prediction. Only the indicated variable was changed while all others were kept constant. The variable was changed from 0 to 10,000 mean fluorescent intensity (MFI). The influence on positive prediction using probability was evaluated. HLA class I (dashed lines) or class II (solid lines) antibodies impact on ML crossmatch prediction are shown in FIG. 3B. The vertical line represents the institutional unacceptable threshold (3,000 MFI). The horizontal line represents the traditional ML model predictive positivity cutoff value of 0.5. HLA class I antibodies were assessed using T cells and HLA class II antibodies were assessed using B cells.

### HLA Antibody Model Performance

To ensure that the ML model scores were consistent with basic transplant immunobiology, we evaluated the ML model performance on T cell prediction using HLA class II antibody data. Since T cells lack HLA class II expression, HLA class II data should not affect model predictions. Using only HLA class II data yielded a low average positive crossmatch probability of 0.001.

A comparison of sensitivity (Sens), specificity (Spec), negative predictive value (NPV), positive predictive value (PPV), and likelihood ratios (LR) was performed between the current manual expert-assigned VXM, summed MFI thresholding approach (Weimer and Newhall 2019), and the HLA antibody ML model. A summary is shown in Table 2. The current expert VXM had excellent specificity and NPV from the ease of predicting negative FCXM when no HLA antibodies are present. However, the expert VXM had low sensitivity and PPV. The ML model slightly outperformed the summed MFI thresholding approach for NPV and sensitivity. While the thresholding approach had a higher PPV, consistent with applying a conservative threshold (Table 2). Specificity was equivalent across all the approaches evaluated.

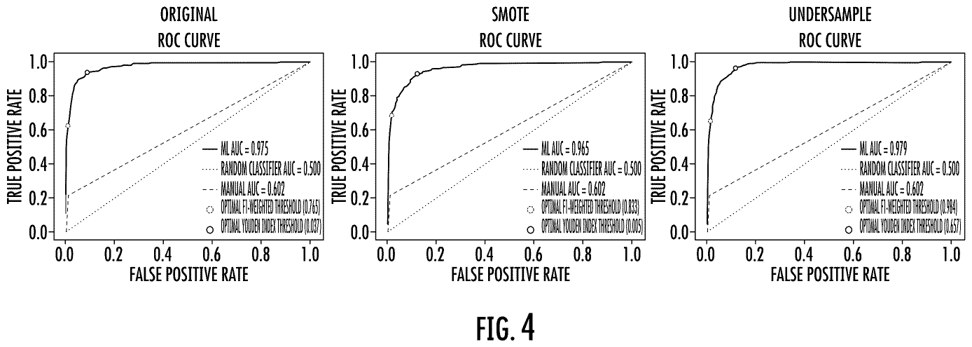

To evaluate the diagnostic ability of each model to predict crossmatch outcomes, receiver-operating-characteristic (ROC) curves and the area under the curve (AUC) were determined (FIG. 4). The AUC for the HLA antibody ML model was 0.975 compared to the manual VXM AUC of 0.602 (FIG. 4). Importantly, all the techniques (SMOTE and Undersampling) to address the data imbalance did not significantly alter the model performance (AUC: 0.965-0.979). These data strongly indicate that, using optimized prediction thresholds, the HLA antibody ML model can accurately predict crossmatch outcomes with minimal human interaction.

FIG. 4 illustrates ML model performance. ROC analysis using either unmanipulated original data (left), SMOTE (center), or undersampling (right). The blue line represents the ROC curve for that model. The dashed line represents the manual VXM process, and the dotted line represents random ROC performance. The blue dot represents the optimal f1-weighted threshold while the green dot represents the optimal Youden index threshold.

TABLE 2
ML model diagnostic performance metrics.

| | | | Machine | Machine |
|---|---|---|---|---|
| | | Summed MFI | Learning: HLA | Learning: HLA |
| | Expert | Thresholding | Antibody | Antibody |
| | VXM | Model | (Original) | (SMOTE) |
| PPV | 0.490 | 0.931 | 0.858 | 0.778 |
| NPV | 0.970 | 0.961 | 0.973 | 0.977 |
| Spec | 0.980 | 0.995 | 0.993 | 0.986 |
| Sen | 0.385 | 0.606 | 0.726 | 0.735 |
| LR+ | 19.25 | 121.20 | 103.71 | 52.50 |
| LR− | 0.628 | 0.396 | 0.276 | 0.269 |

PPV; positive predictive value. NPV; negative predictive value. Spec; specificity. Sens; sensitivity. LR+, positive likelihood ratio. LR−, negative likelihood ratio.

### Discussion

As the demand for pretransplant immunologic risk assessments has increased, so has the need and requirement for more accurate and rapid tools. Here we describe the development and evaluation of a digital alloimmune risk assessment tool (DARA) to address these challenges that used machine learning models to predict allele-level HLA typing (as needed), gather and analyze DSA, and predict physical crossmatch results (FIG. 1). As stated by previous researchers, accurate prediction of HLA alleles remains a substantial challenge in clinical practice (Engen et al. 2021). With the growth of high-resolution HLA typing for solid organ transplant recipients and donors, the accuracy of the ML model will likely move closer to that observed with the non-imputed dataset (Table 1). Our DARA achieved a ROC-AUC of 0.975, outperforming the current standard of care VXM methods (FIG. 4).

Importantly, the HLA antibody model's ability to maintain high specificity (99.3%) while improving sensitivity suggests that it could reduce the number of unnecessary physical crossmatches without increasing the risk of unexpected positive crossmatches. This may streamline the allocation process, reduce cold ischemia times, and improve graft outcomes (Baxter-Lowe et al. 2014).

A benefit of applying machine learning to pre-transplant immunologic compatibility is the ability to learn complex patterns and reduce the human bias in interpreting them. The current approach to assessing HLA antibodies has numerous established issues (Sullivan et al. 2017; Ellis et al. 2012; Greenshields and Liwski 2019). However, current physical crossmatch assessments rely heavily on human interpretation of complex HLA antibodies to estimate immunologic compatibility and risk (Morris et al. 2010; Ellis et al. 2012; Olszowska-Zaremba et al. 2022). The HLA antibody ML approach outperformed the manual human expert prediction, particularly when predicting positive results (Table 2). Expectedly, experts outperformed the ML model in predicting negative results (Table 2; (Olszowska-Zaremba et al. 2022; Ellis et al. 2012)) given that experts can readily understand the relationship between low or near-zero HLA antibody levels and negative crossmatch results. Given the issues with HLA antibody detection and the observed phenomenon of shared epitope spreading (Garcia-Sanchez et al. 2020; Sullivan et al. 2017; Sullivan et al. 2020), it is not surprising that the HLA antibody model had lower sensitivity performance. Potential ways of addressing this deficiency are cross-reactive epitope group (CREG) analysis, HLA eplet-based analysis, and HLA serotype-based analysis (Norin et al. 2022; Osoegawa et al. 2022).

The HLA antibody model enables understanding of the impact specific HLA antibodies have on crossmatch results. The structure of the HLA antibody scoring matrix likely allows for more generalizability of the ML model across different sites as only a single DSA value is required for each HLA locus. While still locally accurate, HLA antigens or alleles can be used as features for a similar ML model; however, those models have limited practical application due to the lack of depth to each HLA antigen or allele. Importantly, the HLA antibody model has learned which HLA antibodies are commonly considered over-reactive in the antibody detection assay (FIGS. 3A and 3B). In particular, the rank order of the HLA antibodies corresponds to the established HLA antigen expression profile on lymphocytes (Hughes et al. 2020; Badders et al. 2015; Cornaby et al. 2022). For instance, HLA-DQ antibodies required higher MFI values (approximately 6,000) to affect predictions, aligning with known issues of over-reactivity in single antigen bead assays. Lastly, the HLA match status of the potential pair is not directly evaluated or considered by the ML model. For pairs where a recipient and donor are matched at several HLA loci, regardless of the DSA MFI detected, those DSA would be reduced 100-fold when the ML model predicts the likelihood of a positive XM (see Materials and Methods). This process ensures that highly matched pairs will appropriately be handled by the ML model when considering DSA MFI at the predictor.

The current practice of transplant centers is to list unacceptable HLA mismatches based on a recipient's HLA antibody profile using an MFI cutoff value; however, in many cases, this cutoff value is applied across all HLA loci rather than individualized to specific HLA loci. The HLA antibody model enables a deeper understanding of which HLA antibodies should be avoided given a recipient's medical status and how best to manage the associated immunologic risk. Consistent with published reports (Bettinotti et al. 2016; Raghavan et al. 2019), higher levels of HLA-DQ antibodies were necessary to induce a positive crossmatch prediction (FIG. 3B). Interestingly, antibodies targeting HLA-C did not induce a positive crossmatch prediction even at high levels. This observation may be from the linkage disequilibrium between HLA-B and HLA-C, and FIG. 3B shows the model places more importance on antibodies targeting HLA-B. The reduced impact of HLA-C antibodies on crossmatch prediction is consistent with the over-representation of HLA-C and the over-reactivity of some HLA-DQ proteins on the solid phase antibody detection assay compared to their biological expression.

We addressed crossmatch outcome imbalance in the data using techniques such as SMOTE and random undersampling but did not find significant improvement in the model's performance. The AUC remained consistently high (0.975-0.979) across all approaches (FIG. 4). This resilience to class imbalance is likely due to several factors including threshold optimization and inherent data characteristics. It is likely the original data may have captured the important patterns distinguishing positive and negative crossmatches, even with the outcome imbalance. This suggests that the features used for model prediction are highly informative for crossmatch prediction.

Our study has several limitations. The allele prediction model impacts all downstream analyses. While the ML models are more robust than traditional methods, they have not been hardcoded to understand if the recipient is non-sensitized (no HLA antibodies) leading to a negative crossmatch. Additionally, the model has been trained and tested using internal data; validation through external data would enhance the confidence in the model prediction.

The work shown here is the initial step to a larger integration of data into the pre-transplant immunologic risk assessment that may include repeat HLA and eplet mismatches, HLA antibody quality, and other relevant parameters.

### Materials and Methods

### Study Design

Flow cytometric crossmatches (FCXM) performed at UNC Health from January 2016 to May 2024 were analyzed. Initially, 16,061 FCXM results were extracted from the laboratory information system (HistoTrac, version 2.52.9). After removing known false-negative, false-positive, inconclusive, incomplete, autologous, and uninterpretable FCXM, 13,158 cases (12,354 negative and 804 positive) remained for analysis.

Recipient and donor HLA typing data were obtained from HistoTrac. HLA antibody data from January 2013 to May 2024 were extracted from HLA Fusion (version 4.2, OneLambda). HLA antibody mean fluorescence intensity (MFI) was determined using the OneLambda single antigen bead assay, performed as previously described (Weimer and Newhall 2019).

FCXM procedures used pronase-treated lymphocytes, with positive cutoffs determined using normal human serum according to established laboratory practices (Weimer and Newhall 2019). All data were de-identified and processed using Python (version 3.8.15) in local and Microsoft Azure Cloud environments.

### Machine Learning (ML) Datasets and Training

The analysis included:
13,158 unique flow cytometric crossmatches
1,903 unique recipients
2,513 unique donors
5,703 unique sera
29,070 HLA typing data points at the highest available resolution for all HLA loci

Data were split 75/25 for training and testing. Multiple supervised learning models were tested including Random Forest, XGBoost, Balanced Random Forest, Random Undersampling Classifier, and Logistical Regression Classifier with the best chosen using a custom-designed training metric. This metric provided differential penalties for false positive and false negative predictions, emphasizing the reduction of false negatives. The model was further optimized by determining the threshold that maximized the f1-weighted score, which balances precision and recall for imbalanced datasets. Examples of machine learning model hyperparameters for the XGBoost and Random Forest Classifiers used to implement the HLA antibody model and for a Random Forest Classifier used to implement the donor allele imputation model are provided below the section labeled “Example Machine Learning Model Hyperparameters”.

HLA antibody profiles were obtained directly from HLA Fusion (version 4.2). Donor-specific antibodies (DSA) were automatically determined by matching donor HLA alleles to recipient HLA antibodies in the single-antigen bead (SAB) assay. When a donor HLA allele was not represented in the SAB testing, a dictionary of HLA alleles and their SAB equivalents (generated by amino acid alignment using BioPython version 1.76) was used to select the closest match. For DQ and DP, the most likely donor heterodimer was determined using global DQA1-DQB1 haplotype frequency and DPA1-DPB1 haplotype frequency. The highest DSA MFI was used for each HLA locus. Any DSA that was also a recipient-specific (i.e., donor and recipient are an allele level match) antibody was reduced 100-fold before model training. The recipient's current calculated panel reactive antibody (cPRA) was also included as an ML feature, with missing values imputed using the mean cPRA of the entire dataset. If a donor lacked all DRB3/4/5, an MFI value of minus 1 (−1) was used. HLA allele prediction.

Eight separate XGBoost models (one for each major HLA locus) were trained to predict HLA alleles when not previously known. These models used self-identified Race as a feature for allele prediction. For class I models, HLA antigen level data were used as inputs to predict a single HLA allele. HLA-DPB1 was not predicted, the allele for HLA-DPB1 was presumed to be the most common allele from real-time PCR-detected alleles. HLA-DPB1 was typed using LinkSeq HLA-ABCDRDQDP SABR 384 typing kit (One Lambda). Only missing alleles were predicted; known alleles were used directly in ML training and evaluation for crossmatch prediction.

Each imputation model was trained on high-resolution HLA genotyping data at two-field resolution that was converted to HLA serologic equivalents for training and evaluation. The training dataset varied by HLA locus and ranged from 16,164-60,072 high-resolution patients. The evaluation dataset of each model was HLA locus dependent and ranged from 4,041-15,019. The data were split 80% for training and 20% for testing. Each model was generated using five-fold cross-validation.

### Crossmatch ML Training and Assessment

Data were prepared by predicting missing donor and recipient HLA information using the ML models. Features were created using the highest donor specific HLA antibody MFI and accounted for DQ and DP heterodimer formation given the donor and recipient HLA genotypes, with cell type (T-cell or B-cell) treated as one of the features, allowing the model to predict both simultaneously. Race was not included as a feature for crossmatch prediction.

Model performance was assessed using true positives (TP), false positives (FP), true negatives (TN), and false negatives (FN) against flow cytometry crossmatch results. Precision (TP/(TP+FP)) and recall (TP/(TP+FN)) were calculated.

To address the imbalanced nature of the dataset, two techniques were applied:
1. Synthetic Minority Over-sampling Technique (SMOTE): The minority class (positive XM cases) was oversampled to equal the negative XM cases in the training data;
2. Random undersampling: The majority class (negative XM cases) was down sampled in the training data; and
   ML model evaluation was always performed using the unmanipulated original dataset.

### Individual HLA Antibody Impact on ML Prediction Probability

To evaluate the relative importance and impact of individual HLA antibodies on positive crossmatch outcomes, we employed Partial Dependence Plots (PDPs). PDPs are a model-agnostic method that helps visualize the marginal effect of a single feature on the predicted outcome of a machine-learning model while accounting for the average effects of all other features.

For each HLA antibody feature in our trained model: a. a range of MFI values was created, starting from the minimum observed in the dataset up to either the maximum observed value or 10,000 MFI, whichever was lower. b. This range was divided into equally spaced intervals (e.g., 100 points) to create a smooth curve.

For each point in this MFI range: a. The entire dataset was copied, replacing the original MFI value for the antibody of interest with the current point value. b. Predictions were made using our trained model on these modified datasets. c. The average predicted probability of a positive crossmatch was calculated across all these predictions.

The resulting curve shows how the predicted probability of a positive crossmatch changes as the MFI of a specific HLA antibody varies, while averaging out the effects of all other features. This process was repeated for each HLA antibody feature in our model.

### ML Feature Permutation Determination

To assess the relative importance of features in our model, we employed permutation importance, a model-agnostic method that measures the impact of each feature on model performance. This technique operates by randomly shuffling the values (HLA antibody MFIs) of a single feature while keeping all others constant, then evaluating the change in the model's performance metric. The process is repeated for each feature independently. The permutation process was repeated 10 times for each feature to obtain a distribution of importance scores, enhancing the reliability of our estimates. The analysis was conducted after fitting the model to the entire dataset, providing a comprehensive view of feature importance across all available data.

### Detection Error Tradeoff (DET) and Precision-Recall Curves

The DET curve was generated to visualize the tradeoff between false negative rate (FNR) and false positive rate (FPR) across various decision thresholds. The model's predicted probabilities for the test set were obtained. These probabilities were compared against a range of thresholds to compute FNR and FPR pairs. The curve was plotted with FPR on the x-axis and FNR on the y-axis. Lower curves indicate better model performance, with the optimal point balancing FNR and FPR according to our specific clinical requirements.

The Precision-Recall curve was used to assess the model's performance, particularly in the context of the imbalanced dataset. For both curves, the optimal threshold was determined before these analyses using a custom metric that balanced sensitivity and specificity according to clinical priorities. This threshold was applied consistently across all evaluations to ensure coherence between model training, validation, and performance visualizations.

### Thresholding Model Metrics

For the same set of donors and patients used in crossmatch ML training, total class I and class II donor-specific HLA antibody MFI were calculated from patient SAB data. Thresholds applied to the sum of the MFI values were tested in increments of 10, seeking to maximize the number of TP+TN. Values above the threshold were predicted positive, and values below were predicted negative. The optimal threshold for T-cell crossmatches (using class I antibody totals) was 4990, and for B-cell crossmatches (using class I plus class II antibody totals) was 9350.

### Expert VXM

1,751 VXM were performed at UNC Health from January 2016 to February 2024 and were exported from HistoTrac. Equivocal VXM interpretations were removed. Data were connected to FCXM by unique identifiers. FCXM cases with inconclusive, incomplete, and uninterpretable results were removed.

### Statistics

Scikit-learn (version 1.2.0) was used for ML training, testing, and metrics. XGBoost (version 1.6.1), imblearn (version 0.10.1), and optuna (version 4.0.0) were used for ML training, testing, and evaluation purposes.

### Cross-Match Prediction Example and Exemplary System Architecture

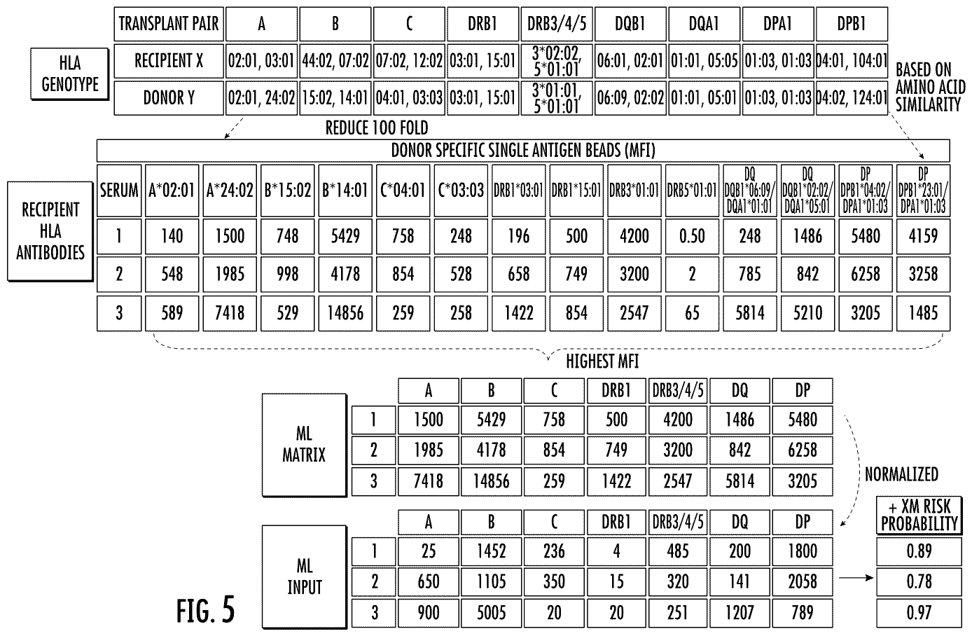

FIG. 5 illustrates an example of HLA antibody model inputs and outputs for a potential transplant recipient X and a donor Y. In FIG. 5, the uppermost table includes the HLA genotype data for the potential transplant recipient X and the donor Y. The HLA genotype data identifies HLA genes of the recipient and the donor. The HLA genotype data can be obtained from gene sequencing of HLA samples of the recipient and the donor or by imputing the HLA genes of the recipient and the donor by inputting donor or recipient serology data into the allele imputation models described above, which output predicted alleles of the donor or the recipient.

The central table in FIG. 5 illustrates DSA MFI values obtained from mixing donor specific single antigen beads coated with HLA antigens with three different serum (blood) samples of the recipient and measuring the mean fluorescence intensity using flow cytometry testing. Each column in the table represents DSA MFI values for a single HLA gene. Adjacent columns represent DSA MFI values for HLA gene pairs. For each HLA gene pair, the highest DSA MFI value is selected for each serum sample. For example, using the A*02:01 and A*24:02 gene pair as an example, the highest of the two DSA MFI values for serum 1 is 1500, so 1500 is selected, as indicated by row 1, column 1 in the ML matrix table. The highest MFI values for each gene pair are normalized. The normalized DSA MFI values are illustrated by the table labeled ML input. The normalized DSA MFI values are then input to the HLA antibody model, which generates, as output, a positive crossmatch probability value for each serum sample of the recipient. The positive crossmatch probability value indicates a likelihood of an unsuccessful transplant outcome.

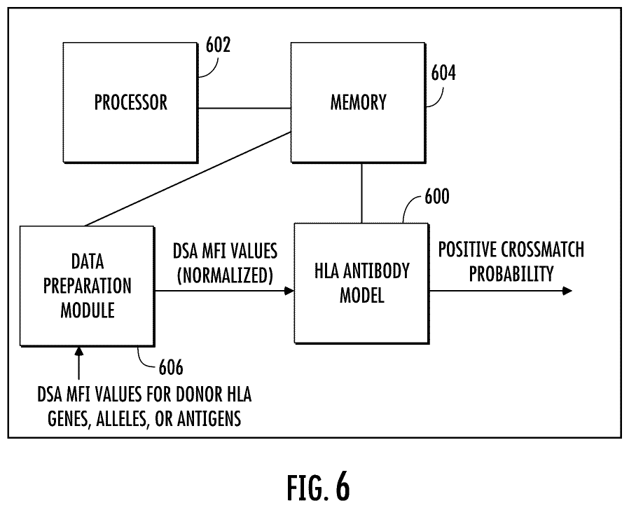

FIG. 6 is a block diagram illustrating an example of a trained machine learning model and its use to generate crossmatch probability values. Referring to FIG. 3, an HLA antibody model 600 may execute on a computing platform including at least one processor 602 and memory 604. HLA antibody model 600 may receive, as input, the normalized MFI values obtained from donor-specific single antigen bead testing as described above and may output the predicted positive crossmatch probability values. A data preparation module 606 may receive, as input, DSA MFI values for donor-specific HLA gene pairs, alleles, or antigens obtained from mixing donor-specific single antigen beads with serum samples of a prospective transplant recipient, for each gene pair, select a highest DSA MFI value, normalize the selected highest DSA MFI values, and input the normalized DSA MFI values into HLA antibody model 600. HLA antibody model 600 and data preparation module 606 may be implemented using computer executable instructions stored in memory 604 and executed by processor 602.

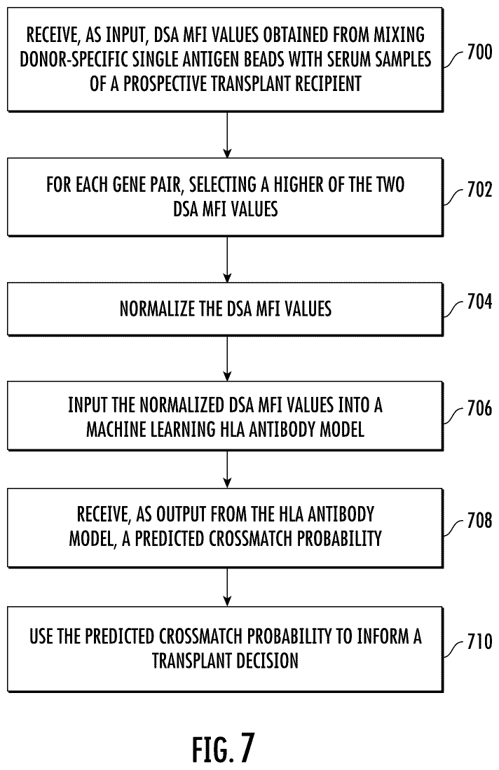

FIG. 7 is a flow chart illustrating an exemplary process for predicting a virtual crossmatch outcome using a machine learning HLA antibody model. Referring to FIG. 7, in step 700, the process includes receiving, as input, DSA MFI values obtained from mixing donor-specific single antigen beads with serum samples of a prospective transplant recipient. In one example, the DSA MFI values are for each gene of each HLA gene pair of the donor. In another example, the DSA MFI values are DSA MFI values for alleles of the HLA genes of the donor. In yet another example, the DSA MFI values are MFI values for antigens encoded by the HLA genes of the donor. For the case where the DSA MFI values are MFI values for genes in each HLA gene pair of the donor, HLA gene pairs of the donor may be determined either through DNA sequencing or through imputation using one of the imputation models described above. For each HLA gene pair of the donor, DSA single antigen beads are mixed with serum of the recipient, and MFI values are measured using flow cytometry. Each single antigen bead is coated with an antigen encoded by a specific HLA allele (A*02:01, DRB1*04:01), and the alleles can be converted into serologic equivalents. The serologic equivalents are the antigens.

Recipient HLA genotype is used in the model as a reduction factor for the MFI value. A recipient and donor may match HLA genotypes for any gene, if they do match meaning donor HLA for a gene equals recipient HLA for that gene the donor specific MFI value is reduced 100-fold. This is done to reduce self-reactive (recipient specific antibodies) antibodies on the model performance.

In step 702, the process further includes, for each gene pair, selecting a higher of the two DSA MFI values. For example, for each donor-specific HLA gene pair, the higher of the two DSA MFI values is selected because the higher value indicates that the recipient is more immune-reactive to the donor HLA gene and therefore more likely to reject a transplant. Alternatively, if the DSA MFI values are for alleles or antigens, step 702 can be omitted, as there will be only one DSA MFI value for each allele or antigen.

In step 704, the process further include normalizing DSA MFI values. For example, the DSA MFI values selected in step 702 or the DSA MFI values of alleles or antigens received in step 700 may be normalized according to the input range allowed by the HLA antibody model. Our ML model uses RobustScaler to convert the MFI values into a normalized value. Robust Scaler scales data based on the interquartile range without using the median value. This happens per input feature. The recipient's cPRA is normalized using MinMax. So scaled 0-1 as the numbers are always between 0-100%.

In step 706, the process further includes inputting the normalized DSA MFI values into the HLA antibody model. For example, normalized MFI values, such as those illustrated in the ML input table, may be input into the machine learning HLA antibody model. The machine learning HLA antibody model is trained as described herein using known transplant outcome data. The input features used to train the HLA antibody model may be aggregate DSA MFI values, as described herein. Alternatively the input features used to train the HLA antibody model may be reorganized to be HLA alleles (A*02:01, etc.) or HLA antigens A*02, etc.) for ML training. These can lead to insights into how influential particular HLA antibodies either allele-level or antigen-level are on crossmatch outcomes and thus what should be considered unacceptable in the organ procurement and transport network (OPTN) system.

In step 708, the process further includes receiving, as output from the HLA antibody model, a predicted crossmatch probability. For example, the HLA antibody model will output predicted positive crossmatch probability value that indicates a likelihood that a transplant will be rejected by the prospective recipient.

In step 710, the process further includes using the predicted crossmatch probability to inform a transplant decision. For example, a physician may use the positive transplant probability value as a factor in determining whether or not to perform an organ transplant for a particular donor-recipient pair.

### Example Machine Learning Model Hyperparameters

The following are example hyperparameters searched over for the HLA antibody model that performs the crossmatch predication:

The set of hyperparameters that are searched over:

param_dict = {‘RandomForestClassifier’: {
‘preprocessor_{——}num’:
[QuantileTransformer(random_state=56)],
‘classifier_{——}max_depth’: [10, 15, 20],
‘classifier_{——}max_features': [‘sqrt’, ‘log2’],
‘classifier_{——}n_estimators': [500, 750, 1000],
‘classifier_{——}oob_score’: [True],
‘classifier_{——}class_weight’: [{0:1, 1:5}, {0:1, 1:3},
{0:1, 1:8}], ‘classifier_{——}random_state’:[56]},
‘GradientBoostClassifier’: {
‘classifier_{——}max_depth’: [3, 6],
‘classifier_{——}n_estimators': [200,300,500],
‘classifier_{——}learning_rate’: [0.1, 0.05],
‘classifier_{——}subsample’: [ 0.8 ]},
‘XGBClassifier’: {
‘classifier_{——}max_depth’: [10, 15, 20],
‘classifier_{——}n_estimators': [200, 500, 1000],
‘classifier_{——}min_child_weight’: [1, 2, 3],
‘classifier_{——}gamma’: [ 0.1, 0.5 ],
‘classifier_{——}learning_rate’: [0.05, 0.01],
‘classifier_{——}colsample_bytree’: [ 0.8 ],
‘classifier_{——}scale_pos_weight’: [8],
‘classifier_{——}early_stopping_rounds': [75],
‘classifier_{——}lambda’:[1, 2, 3]}, #
‘BalancedRandomForestClassifier’: {
‘preprocessor_{——}num’:
[QuantileTransformer(random_state=56), RobustScaler( ),
MinMaxScaler(feature_range= (0, 10))],
‘classifier_{——}max_depth’: [2, 3, 4],
‘classifier_{——}max_features': [‘sqrt’, ‘log2’],
‘classifier_{——}n_estimators': [100, 200, 500],
‘classifier_{——}oob_score’: [True],
‘classifier_{——}class_weight’: [‘balanced_subsample’],
‘classifier_{——}random_state’:[56],
‘classifier_{——}sampling_strategy’:[‘majority’]},
}

The following are hyperparameters that resulted from the search for the XGBoost and Random Forest models used to implement the HLA antibody model.

XGBoost

Model Hyperparameters:
memory: None

| | |
|---|---|
| steps: | [(‘cell_type_selector’, |
| CellTypeFeatureSelector( )), | (‘preprocessor’, |

ColumnTransformer(remainder=‘passthrough’,
transformers=[(‘num’,
QuantileTransformer( ),

[‘B’, ‘DP’, ‘DRB345’,

‘A’, ‘DQ’, ‘DRB1’, ‘C’]),

(‘num_pra’,

MinMaxScaler( ), [‘cpra’])],
verbose_feature_names_out=False)),

| | |
|---|---|
| (‘classifier’, | XGBClassifier(base_score=None, |

```
booster=None, callbacks=None,
    colsample_bylevel=None,
colsample_bynode=None,
    colsample_bytree=0.9113013754001089,
device=None,
    early_stopping_rounds=None,
enable_categorical=False,
    eval_metric=‘aucpr’, feature_types=None,
    gamma=0.15386577449595482,
grow_policy=None, importance_type=None,
    interaction_constraints=None,
learning_rate=0.034394408363265794,
```

| | |
|---|---|
| max_bin=None, | max_cat_threshold=None, |

max_cat_to_onehot=None,

| | |
|---|---|
| max_delta_step=None, | max_depth=7, |

max_leaves=None,

| | |
|---|---|
| min_child_weight=2, | missing=nan, |

monotone_constraints=None,

| | |
|---|---|
| multi_strategy=None, | n_estimators=417, |

n_jobs=None,

| | |
|---|---|
| num_parallel_tree=None, | random_state=42, |

```
...))]
  transform_input: None
  verbose: False
  cell_type_selector: CellTypeFeatureSelector( )
  preprocessor:
ColumnTransformer(remainder=‘passthrough’,
      transformers=[(‘num’,
QuantileTransformer( ),
```

[‘B’, ‘DP’, ‘DRB345’,

‘A’, ‘DQ’, ‘DRB1’, ‘C’]),

(‘num_pra’,

MinMaxScaler( ), [‘cpra’])],
verbose_feature_names_out=False)

| | |
|---|---|
| classifier: | XGBClassifier(base_score=None, |

```
booster=None, callbacks=None,
    colsample_bylevel=None,
colsample_bynode=None,
    colsample_bytree=0.9113013754001089,
device=None,
    early_stopping_rounds=None,
enable_categorical=False,
    eval_metric=‘aucpr’, feature_types=None,
    gamma=0.15386577449595482,
grow_policy=None, importance_type=None,
    interaction_constraints=None,
learning_rate=0.034394408363265794,
```

| | |
|---|---|
| max_bin=None, | max_cat_threshold=None, |

max_cat_to_onehot=None,

| | |
|---|---|
| max_delta_step=None, | max_depth=7, |

max_leaves=None,

| | |
|---|---|
| min_child_weight=2, | missing=nan, |

monotone_constraints=None,

| | |
|---|---|
| multi_strategy=None, | n_estimators=417, |

n_jobs=None,

| | |
|---|---|
| num_parallel_tree=None, | random_state=42, |

```
...)
  cell_type_selector_{——}feature_pattern: \hat{ }[ABC](\d+)?$
  preprocessor_{——}force_int_remainder_cols: deprecated
  preprocessor_{——}n_jobs: None
  preprocessor_{——}remainder: passthrough
  preprocessor_{——}sparse_threshold: 0.3
  preprocessor_{——}transformer_weights: None
```

| | |
|---|---|
| preprocessor_{——}transformers: | [(‘num’, |

```
QuantileTransformer( ), [‘B’, ‘DP’, ‘DRB345’, ‘A’, ‘DQ’,
‘DRB1’, ‘C’]), (‘num_pra’, MinMaxScaler( ), [‘cpra’])]
  preprocessor_{——}verbose: False
  preprocessor_{——}verbose_feature_names_out: False
  preprocessor_{——}num: QuantileTransformer( )
  preprocessor_{——}num_pra: MinMaxScaler( )
  preprocessor_{——}num_{——}copy: True
  preprocessor_{——}num_{——}ignore_implicit_zeros: False
  preprocessor_{——}num_{——}n_quantiles: 1000
  preprocessor_{——}num_{——}output_distribution: uniform
  preprocessor_{——}num_{——}random_state: None
  preprocessor_{——}num_{——}subsample: 10000
  preprocessor_{——}num_pra_{——}clip: False
  preprocessor_{——}num_pra_{——}copy: True
  preprocessor_{——}num_pra_{——}feature_range: (0, 1)
  classifier_{——}objective: binary:logistic
  classifier_{——}base_score: None
  classifier_{——}booster: None
  classifier_{——}callbacks: None
  classifier_{——}colsample_bylevel: None
  classifier_{——}colsample_bynode: None
  classifier_{——}colsample_bytree: 0.9113013754001089
  classifier_{——}device: None
  classifier_{——}early_stopping_rounds: None
  classifier_{——}enable_categorical: False
  classifier_{——}eval_metric: aucpr
  classifier_{——}feature_types: None
  classifier_{——}gamma: 0.15386577449595482
  classifier_{——}grow_policy: None
  classifier_{——}importance_type: None
  classifier_{——}interaction_constraints: None
  classifier_{——}learning_rate: 0.034394408363265794
  classifier_{——}max_bin: None
  classifier_{——}max_cat_threshold: None
  classifier_{——}max_cat_to_onehot: None
  classifier_{——}max_delta_step: None
  classifier_{——}max_depth: 7
  classifier_{——}max_leaves: None
  classifier_{——}min_child_weight: 2
  classifier_{——}missing: nan
  classifier_{——}monotone_constraints: None
  classifier_{——}multi_strategy: None
  classifier_{——}n_estimators: 417
  classifier_{——}n_jobs: None
  classifier_{——}num_parallel_tree: None
  classifier_{——}random_state: 42
  classifier_{——}reg_alpha: 0.6306229098947158
  classifier_{——}reg_lambda: 0.4809373456951124
  classifier_{——}sampling_method: None
  classifier_{——}scale_pos_weight: 16.70001751090224
  classifier_{——}subsample: 0.7986553771175036
  classifier_{——}tree_method: None
  classifier_{——}validate_parameters: None
  classifier_{——}verbosity: None
  classifier_{——}use_label_encoder: False
```

Random Forest

| Model Hyperparameters: | |
|---|---|
| memory: None | |
| steps:[(‘cell_type_selector’,CellTypeFeatureSelector( )), | |
| (‘preprocessor’, ColumnTransformer(transformers=[(‘num’, | |
| Pipeline(steps=[(‘scaler’, | |
| RobustScaler( ))]), | |
| | [‘A’, ‘DP’, ‘C’, ‘DRB1’, |
| ‘DQ’, ‘DRB345’, ‘B’]}, | |
| | (‘num_pra’, |
| MinMaxScaler( ), [‘cpra’])], | |
| verbose_feature_names_out=False)), | |
| (‘classifier’, | RandomForestClassifier(bootstrap=False, |
| class_weight=‘balanced_subsample’, | |
| max_depth=22, | |
| max_features=‘log2’, min_samples_leaf=5, | |
| min_samples_split=20, | |
| n_estimators=169, n_jobs=−1; | |
| random_state=42))] | |
| transform_input: None | |
| verbose: False | |
| cell_type_selector: CellTypeFeatureSelector( ) | |
| preprocessor: ColumnTransformer(transformers=[(‘num’, | |
| Pipeline(steps=[{‘scaler’, RobustScaler( ))}); | |
| | [‘A’, ‘DP’, ‘C’, ‘DRB1’, |
| ‘DQ’, ‘DRB345’, ‘B’]), | |
| | (‘num_pra’, |
| MinMaxScaler( ), [‘cpra’])], | |
| verbose_feature_names_out=False) | |
| classifier: | RandomForestClassifier(bootstrap=False, |
| class_weight=‘balanced_subsample’, | |
| max_depth=22, | |
| max_features=‘log2’, min_samples_leaf=5, | |
| min_samples_split=20, | |
| n_estimators=169, n_jobs=−1, | |
| random_state=42) | |
| cell_type_selector_{——}feature_pattern: \hat{ }[ABC](\d+)?$ | |
| preprocessor_{——}force_int_remainder_cols: deprecated | |
| preprocessor_{——}n_jobs: None | |
| preprocessor_{——}remainder: drop | |
| preprocessor_{——}sparse_threshold: 0.3 | |
| preprocessor_{——}transformer_weights: None | |
| preprocessor_{——}transformers: | [(‘num’, |
| Pipeline(steps=[(‘scaler’, RobustScaler( ))]), [‘A’, ‘DP’, | |
| ‘C’, ‘DRB1’, ‘DQ’, ‘DRB345’, ‘B’]), (‘num_pra’, | |
| MinMaxScaler( ), [‘cpra’])] | |
| preprocessor_{——}verbose: False | |
| preprocessor_{——}verbose_feature_names_out: False | |
| preprocessor_{——}num: | Pipeline(steps=[(‘scaler’, |

```
RobustScaler( ))])
  preprocessor_{——}num_pra: MinMaxScaler( )
  preprocessor_{——}num_{——}memory: None
  preprocessor_{——}num_{——}steps: [(‘scaler’, RobustScaler( ))]
  preprocessor_{——}num_{——}transform_input: None
  preprocessor_{——}num_{——}verbose: False
  preprocessor_{——}num_{——}scaler: RobustScaler( )
  preprocessor_{——}num_{——}scaler_{——}copy: True
  preprocessor_{——}num_{——}scaler_{——}quantile_range: (25.0, 75.0)
  preprocessor_{——}num_{——}scaler_{——}unit_variance: False
  preprocessor_{——}num_{——}scaler_{——}with_centering: True
  preprocessor_{——}num_{——}scaler_{——}with_scaling: True
  preprocessor_{——}num_pra_{——}clip: False
  preprocessor_{——}num_pra_{——}copy: True
  preprocessor_{——}num_pra_{——}feature_range: (0, 1)
  classifier_{——}bootstrap: False
  classifier_{——}ccp_alpha: 0.0
  classifier_{——}class_weight: balanced_subsample
  classifier_{——}criterion: gini
  classifier_{——}max_depth: 22
  classifier_{——}max_features: log2
  classifier_{——}max_leaf_nodes: None
  classifier_{——}max_samples: None
  classifier_{——}min_impurity_decrease: 0.0
  classifier_{——}min_samples_leaf: 5
  classifier_{——}min_samples_split: 20
  classifier_{——}min_weight_fraction_leaf: 0.0
  classifier_{——}monotonic_cst: None
  classifier_{——}n_estimators: 169
  classifier_{——}n_jobs: −1
  classifier_{——}oob_score: False
  classifier_{——}random_state: 42
  classifier_{——}verbose: 0
  classifier_{——}warm_start: False
```

A hyperparameter search was not performed for the allele imputation model. The hyperparameters used for a Random Forest classier used to implement the allele imputation model are as follows:

class sklearn.ensemble.RandomForestClassifier(
n_estimators=100,
*
,
criterion=‘gini’,
max_depth=None,
min_samples_split=2,
min_samples_leaf=1,
min_weight_fraction_leaf=0.0,
max_features=‘sqrt’,
max_leaf_nodes=None,
min_impurity_decrease=0.0,
bootstrap=True,
oob_score=False,
n_jobs=−1,
random_state=None,
verbose=0,
warm_start=False,
class_weight=None,
ccp_alpha=0.0,
max_samples=None,
monotonic_cst=None)

The disclosure of each of the following references is incorporated herein by reference in its entirety.

### REFERENCES

1. Tambur, A. R. et al. Sensitization in transplantation: assessment of risk (STAR) 2017 working group meeting report. Am. J. Transplant. 18, 1604-1614 (2018).
2. Roll, G. R. et al. A Virtual Crossmatch-based Strategy Facilitates Sharing of Deceased Donor Kidneys for Highly Sensitized Recipients. Transplantation 104, 1239-1245 (2020).
3. Eby, B. C. et al. Virtual HLA crossmatching as a means to safely expedite transplantation of imported pancreata. Transplantation 100, 1103-1110 (2016).
4. Amico, P., Hönger, G., Steiger, J. & Schaub, S. Utility of the virtual crossmatch in solid organ transplantation. Curr. Opin. Organ Transplant. 14, 656-661 (2009).
5. Takeda, A. et al. Acute humoral rejection of kidney allografts in patients with a positive flow cytometry crossmatch (FCXM). Clin. Transplant. 14 Suppl 3, 15-20 (2000).
6. Downing, J. The lymphocyte crossmatch by flow cytometry for kidney transplantation. Methods Mol. Biol. 882, 379-390 (2012).
7. Sullivan, H. C. et al. (F) Utility of the physical crossmatch for living donor evaluations in the age of the virtual crossmatch. Hum. Immunol. 79, 711-715 (2018).
8. Liwski, R. S. et al. Rapid optimized flow cytometric crossmatch (FCXM) assays: The Halifax and Halifaster protocols. Hum. Immunol. 79, 28-38(2018).
9. Morris, G. P., Phelan, D. L., Jendrisak, M. D. & Mohanakumar, T.

Virtual crossmatch by identification of donor-specific anti-human leukocyte antigen antibodies by solid-phase immunoassay: a 30-month analysis in living donor kidney transplantation. Hum. Immunol. 71, 268-273 (2010).
10. Morris, A. B., Sullivan, H. C., Krummey, S. M., Gebel, H. M. & Bray, R. A. Out with the old, in with the new: Virtual versus physical crossmatching in the modern era. HLA 94, 471-481 (2019).
11. Weimer, E. T. & Newhall, K. A. Development of data-driven models for the flow cytometric crossmatch. Hum. Immunol. 80, 983-989 (2019).
12. Norin, A. J. et al. Determination of unacceptable antigens by summation of anti-HLA eplet antibody strength (MFI) based on single antigen bead assays: Excellent correlation with negative cell based cross matches. Hum. Immunol. 83, 482-493 (2022).
13. Valentin, M. O. et al. Improving the access of highly sensitized patients to kidney transplantation from deceased donors: the spanish PATHI program with allocation based on the virtual crossmatch. Transplantation 108, 787-801 (2024).
14. Wrenn, S. M. et al. Improving the performance of virtual crossmatch results by correlating with nationally-performed physical crossmatches: Obtaining additional value from proficiency testing activities. Hum. Immunol. 79, 602-609 (2018).
15. Johnson, C. P. et al. Renal Transplantation With Final Allocation Based on the Virtual Crossmatch. Am. J. Transplant. 16, 1503-1515 (2016).
16. Perssaari, J. P., Jaatinen, T. & Merenmies, J. Donor-specific HLA antibodies in predicting crossmatch outcome: Comparison of three different laboratory techniques. Transpl. Immunol. 46, 23-28 (2018).
17. Piazza, A. et al. Virtual crossmatch in kidney transplantation. Transplant. Proc. 46, 2195-2198 (2014).
18. Chowdhry, M., Agrawal, S., Thakur, Y., Guleria, S. & Sharma, V. Implication of a positive virtual crossmatch with negative flow crossmatch: A mind-boggler. Asian J. Transfus. Sci. 14, 79-82 (2020).
19. Olszowska-Zaremba, N., Zagożdżon, R. & Gozdowska, J. Accuracy of virtual crossmatch (VXM) prediction of physical crossmatch (PXM) results of donor specific antibody (DSA) in routine pretransplant settings-a single-center experience. Transpl. Immunol. 72, 101583 (2022).
20. Badders, J. L., Jones, J. A., Jeresano, M. E., Schillinger, K. P. & Jackson, A. M. Variable HLA expression on deceased donor lymphocytes: Not all crossmatches are created equal. Hum. Immunol. 76, 795-800 (2015).
21. Montgomery, M. C., Liu, C., Petraroia, R. & Weimer, E. T. Using Nanopore Whole-Transcriptome Sequencing for Human Leukocyte Antigen Genotyping and Correlating Donor Human Leukocyte Antigen Expression with Flow Cytometric Crossmatch Results. J. Mol. Diagn. 22, 101-110 (2020).
22. Miller, R. J. H. et al. Temporal shift and predictive performance of machine learning for heart transplant outcomes. J. Heart Lung Transplant. 41, 928-936 (2022).
23. Tian, D. et al. Machine Learning-Based Prognostic Model for Patients After Lung Transplantation. JAMA Netw. Open 6, e2312022 (2023).
24. Mark, E., Goldsman, D., Gurbaxani, B., Keskinocak, P. & Sokol, J. Using machine learning and an ensemble of methods to predict kidney transplant survival. PLoS ONE 14, e0209068 (2019).
25. Becker, J. U. et al. Artificial intelligence and machine learning in nephropathology. Kidney Int. 98, 65-75 (2020).
26. Senev, A., Emonds, M.-P. & Naesens, M. Second field high-resolution HLA typing for immunologic risk stratification in kidney transplantation. Am. J. Transplant. 21, 3502-3503 (2021).
27. Chawla, N. V., Bowyer, K. W., Hall, L. O. & Kegelmeyer, W. P. SMOTE: Synthetic Minority Over-sampling Technique. jair 16, 321-357 (2002).
28. Shao, H., Liu, X., Zong, D. & Song, Q. Optimization of diabetes prediction methods based on combinatorial balancing algorithm. Nutr. Diabetes 14, 63 (2024).
29. Engen, R. M., Jedraszko, A. M., Conciatori, M. A. & Tambur, A. R. Substituting imputation of HLA antigens for high-resolution HLA typing: Evaluation of a multiethnic population and implications for clinical decision making in transplantation. Am. J. Transplant. 21, 344-352 (2021).
30. Baxter-Lowe, L. A., Cecka, M., Kamoun, M., Sinacore, J. & Melcher, M. L. Center-defined unacceptable HLA antigens facilitate transplants for sensitized patients in a multi-center kidney exchange program. Am. J. Transplant. 14, 1592-1598 (2014).
31. Sullivan, H. C., Liwski, R. S., Bray, R. A. & Gebel, H. M. The road to HLA antibody evaluation: do not rely on MFR. Am. J. Transplant. 17, 1455-1461 (2017).
32. Ellis, T. M. et al. Diagnostic accuracy of solid phase HLA antibody assays for prediction of crossmatch strength. Hum. Immunol. 73, 706-710 (2012).
33. Greenshields, A. L. & Liwski, R. S. The ABCs (DRDQDPs) of the prozone effect in single antigen bead HLA antibody testing: Lessons from our highly sensitized patients. Hum. Immunol. 80, 478-486 (2019).
34. Garcia-Sanchez, C., Usenko, C. Y., Herrera, N. D. & Tambur, A. R.

The shared epitope phenomenon-A potential impediment to virtual crossmatch accuracy. Clin. Transplant. 34, e13906 (2020).
35. Sullivan, H. C., Krummey, S. M., Gebel, H. M. & Bray, R. A. The utility of second single antigen bead assay: Clearing the water or stirring up mud? Hum. Immunol. 81, 663-670 (2020).
36. Osoegawa, K. et al. A new strategy for systematically classifying HLA alleles into serological specificities. HLA 100, 193-231 (2022).
37. Hughes, A. E. O., Montgomery, M. C., Liu, C. & Weimer, E. T. Quantification of Allele-Specific HLA Expression with Nanopore Long-Read Sequencing. Blood 136, 42-43 (2020).
38. Cornaby, C., Montgomery, M. C., Liu, C. & Weimer, E. T. Unique Molecular Identifier-Based High-Resolution HLA Typing and Transcript Quantitation Using Long-Read Sequencing. Front. Genet. 13, 901377 (2022).
39. Bettinotti, M. P., Zachary, A. A. & Leffell, M. S. Clinically relevant interpretation of solid phase assays for HLA antibody. Curr. Opin. Organ Transplant. 21, 453-458 (2016).
40. Raghavan, M., Yarzabek, B., Zaitouna, A. J., Krishnakumar, S. & Ramon, D. S. Strategies for the measurements of expression levels and half-lives of HLA class I allotypes. Hum. Immunol. 80, 221-227 (2019).

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for predicting a virtual crossmatch outcome using a machine learning human leukocyte antigen (HLA) antibody model, the method comprising:
receiving, as input, donor specific antibody (DSA) mean fluorescence intensity (MFI) values obtained from mixing donor-specific single antigen beads with serum samples of a prospective transplant recipient;
normalizing the DSA MFI values;
inputting the normalized DSA MFI values into the machine learning HLA antibody model;
receiving, as output from the HLA antibody model, a predicted crossmatch probability; and
using the predicted crossmatch probability to inform a transplant decision.

2. The method of claim 1 comprising identifying donor-specific HLA gene pairs, where the DSA MFI values are DSA MFI values for the donor-specific HLA gene pairs and further comprising, for each gene pair, selecting a higher of two DSA MFI values.

3. The method of claim 2 wherein identifying the donor-specific HLA gene pairs comprises identifying the donor-specific HLA gene pairs from DNA sequencing of an HLA system of a donor.

4. The method of claim 2 wherein identifying the donor-specific HLA gene pairs comprises imputing the donor-specific HLA gene pairs from HLA serology data of the donor.

5. The method of claim 2 comprising identifying recipient HLA gene pairs.

6. The method of claim 5 comprising reducing DSA MFI values for donor HLA gene pairs that match recipient HLA gene pairs.

7. The method of claim 1 comprising identifying donor-specific HLA alleles and wherein the DSA MFI values are DSA MFI values for the donor-specific HLA alleles.

8. The method of claim 1 comprising identifying donor-specific antigens encoded by donor-specific HLA gene pairs, wherein the DSA MFI values are DSA MFI values for the donor-specific antigens.

9. The method of claim 1 wherein inputting the normalized DSA MFI values into the machine learning HLA antibody model includes inputting the normalized DSA MFI values into a HLA antibody model trained to predict the crossmatch probability from known transplant outcome data.

10. The method of claim 1 wherein receiving the predicted crossmatch probability includes receiving a predicted positive crossmatch probability indicating a likelihood that a transplant will not be successful.

11. The method of claim 1 wherein using the predicted crossmatch probability to inform the transplant decision includes determining whether or not to perform a transplant for a donor-recipient pair based on the predicted crossmatch probability.

12. A system for predicting a virtual crossmatch outcome using a machine learning human leukocyte antigen (HLA) antibody model, the system comprising:
a computing platform including at least one processor and a memory;
a data preparation module implemented by the at least one processor for receiving, as input, donor specific antibody (DSA) mean fluorescence intensity (MFI) values obtained from mixing donor-specific single antigen beads with serum samples of a prospective transplant recipient and normalizing the DSA MFI values; and
an HLA antibody model implemented by the at least one processor for receiving, as input, the normalized DSA MFI values and generating, as output, a predicted crossmatch probability.

13. The system of claim 12 wherein the data preparation module is configured to identify donor-specific HLA gene pairs, where the DSA MFI values are DSA MFI values for the donor-specific HLA gene pairs and the data preparation module is configured to, for each gene pair, selecting a higher of two DSA MFI values.

14. The system of claim 13 wherein the data preparation module is configured to identify the donor-specific HLA gene pairs from DNA sequencing of an HLA system of a donor.

15. The system of claim 13 wherein the data preparation module is configured to identify the donor-specific HLA gene pairs by imputing the donor-specific HLA gene pairs from HLA serology data of the donor.

16. The system of claim 13 wherein the data preparation module is configured to identify recipient HLA gene pairs.

17. The system of claim 16 wherein the data preparation module is configured to reduce DSA MFI values for donor HLA gene pairs that match recipient HLA genes pairs.

18. The system of claim 12 wherein the data preparation module is configured to identify donor-specific HLA alleles and the DSA MFI values are DSA MFI values for the donor-specific HLA alleles.

19. The system of claim 12 wherein the data preparation module is configured to identify donor-specific antigens encoded by donor-specific HLA gene pairs, wherein the DSA MFI values are DSA MFI values for the donor-specific antigens.

20. The system of claim 12 wherein the HLA antibody model is trained to predict the crossmatch probability from known transplant outcome data.

21. The system of claim 12 wherein the predicted crossmatch probability comprises a predicted positive crossmatch probability indicating a likelihood that a transplant will not be successful.

22. A non-transitory computer readable medium having stored thereon executable instructions that when executed by a processor of a computer control the computer to perform steps comprising:
receiving, as input, donor specific antibody (DSA) mean fluorescence intensity (MFI) values obtained from mixing donor-specific single antigen beads with serum samples of a prospective transplant recipient;
normalizing the DSA MFI values;
inputting the normalized DSA MFI values into a machine learning donor-specific human leukocyte antigen (HLA) antibody model;
receiving, as output from the HLA antibody model, a predicted crossmatch probability; and
using the predicted crossmatch probability to inform a transplant decision.
